(19) United States
(12) Patent Application Publication
(10) Pub. No.: US 2019/0380963 A1
(43) Pub. Date: Dec. 19, 2019

(54) LIPOSOME PREPARATION HAVING HIGH-CONTENT CATIONIC LIPID COMPOUND AND USE THEREOF

(71) Applicant: BIOMICS BIOTECHNOLOGIES CO., LTD., Nantong, Jiangsu (CN)

(72) Inventors: Jianxin CHEN, Nantong, Jiangsu (CN); Wei PENG, Nantong, Jiangsu (CN); Tiejun LI, Nantong, Jiangsu (CN); Yan NI, Nantong, Jiangsu (CN); Wenjian FENG, Nantong, Jiangsu (CN)

(21) Appl. No.: 16/092,198

(22) PCT Filed: Apr. 8, 2017
(86) PCT No.: PCT/CN2017/079810

§ 371 Date: Apr. 11, 2019

(30) Foreign Application Priority Data
Apr. 11, 2016 (CN) 201610222630.2

(51) Int. Cl. A61K 9/127; A61K 47/28; A61K 31/713; A61K 47/14; A61K 47/22

(52) U.S. Cl. CPC A61K 9/1272; A61K 47/28; A61K 47/22; A61K 47/14; A61K 31/713

(57) ABSTRACT

A liposome for delivery of nucleic acid is provided herein, comprising cationic lipid compound, a phospholipid and a PEGylated lipid, wherein the cationic lipid compound is in an amount from 50 wt % to 90 wt % and consist of a lipid compound and a cholesterol lipid compound. A nucleic acid liposome formulation comprising the said liposome and the nucleic acid encapsulated by the liposome and the method for preparing the same are provided therein. Increased amount of nucleic acid can be loaded into the nucleic acid liposome formulation as described therein. Thus, at the same dosage, intake of the cationic lipid compound is decreased, thereby decreasing toxicity.

### CROSS REFERENCE TO RELATED APPLICATIONS

This application claims the benefit of, and priority to, Chinese Patent Application Serial No. 201610222630.2, entitled “Liposome Formulation Comprising a Cationic Lipid Compound in High Amount and the Use Thereof”, filed on Apr. 11, 2016, the entire disclosures of which are incorporated by reference.

### FIELD OF THE INVENTION

The present invention relates to a liposome formulation, more particularly, to a liposome formulation comprising a cationic lipid compound in high amount and the use thereof.

### BACKGROUND OF THE INVENTION

In recent ten years, RNA interference (RNAi) is found to be a self protection mechanism having a function for modulating gene expression in organisms. RNAi is a specific gene silence phenomenon mediated by double-stranded RNA (dsRNA), in which specific enzymes involve. RNAi blocks gene expression at transcriptional level, post-transcriptional level and translational level. With the development of RNAi technology, a variety of nucleic acid medicaments designed and developed based on RNAi mechanism enter clinical trial stage. The nucleic acid medicaments for modulating abnormal gene expression will be a promised effective route for treating diseases.

In recent years, a series of nucleic acid medicaments based on RNAi mechanism show well development, which include small interfering RNAs (siRNAs), microRNAs (miRNAs), non-coding RNAs (ncRNAs), antisense RNAs/DNAs, small ligand RNAs (sliRNAs) and small active RNAs (saRNAs) and the like. These nucleic acids exhibit their functions via various gene modulation mechanisms including RNAi. For example, siRNAs and miRNAs modulate expression level of specific genes in cells via RNAi mechanism. After siRNAs or miRNAs go into cells, these double-stranded RNAs are able to combine with specific proteins in cells to form RNA inducing silencing complexes (RISCs). After unwinding of siRNAs in RISCs, they combine with mRNAs through sequence complementary identification and then mRNAs are cutted to achieve down-regulation of the gene expression of mRNAs. RNAi provides a gene therapy by complementing to the target genes and blocking the expression of mRNA encoding protein.

Vector systems based on the liposomes are used to deliver the nucleic acid medicaments to systematically administer siRNAs encapsulated by the cationic liposomes. There are reports about the efficiency of the nucleic acid liposome formulations in vivo and in vitro. However, there are still several problems in application of nucleic acid liposome formulations: 1) lack of effective in vivo delivery systems; 2) low drug loading capacity for the current liposome formulations; and 3) high in vivo toxicity of the nucleic acid liposome formulations.

In order to overcome the above problems, there is a need to provide a liposome formulation comprising a cationic lipid compound in high amount, which can safely deliver nucleic acid medicaments in high amount into body.

### SUMMARY OF THE INVENTION

In one aspect, a liposome for delivery of nucleic acids is provided herein, comprising a cationic lipid compound, a phospholipid and a PEGylated lipid, in which, the cationic lipid compound is in an amount from 50 wt % to 90 wt %, the phospholipid is in an amount from 5 wt % to 10 wt %, and the PEGylated lipid is in an amount from 0 wt % to 10 wt %. The cationic lipid compound consists of a lipid-type lipid compound and a cholesterol lipid compound. The molar ratio of the lipid-type lipid compound to the cholesterol lipid compound ranges from 4:1 to 1:4. In some embodiments, in the liposome for delivery of nucleic acids as described herein, the cationic lipid compound is in an amount from 60 wt % to 90 wt %, or 70 wt % to 90 wt %, or 75 wt % to 90 wt %, or 80 wt % to 90 wt %, or 85 wt % to 90 wt %.

The lipid-type lipid compound as described herein has a chemical structure selected from Formula I, Formula II, Formula III and Formula IV as shown below,

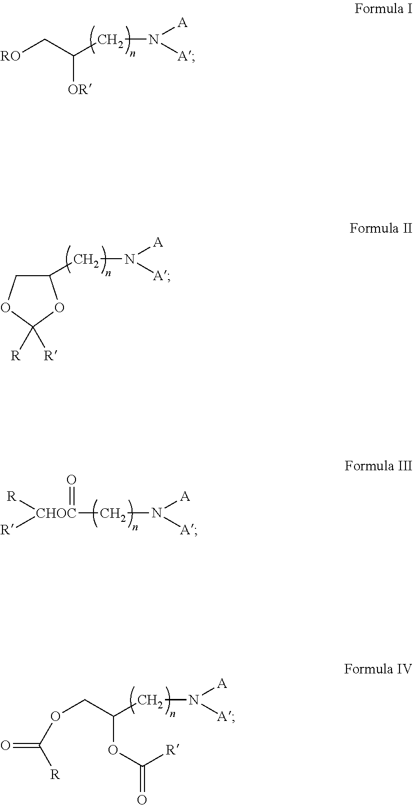

wherein, R and R′ are independently C₁₄-C₂₂ saturated or unsaturated fatty acid, A and A′ are C₁-C₄ alkyl, n=1-4.

In the embodiments as described herein, the compounds having Formula I are seleced from the group consisting of following compounds:
Compound 1: R═R′=linoleic acid group, n=1, A=CH₃, A′=CH₃CH₂;
Compound 2: R═R′=linoleic acid group, n=2, A=CH₃, A′=CH₃CH₂;
Compound 3: R═R′=linoleic acid group, n=3, A=CH₃, A′=CH₃CH₂;
Compound 4: R═R′=linoleic acid group, n=4, A=CH₃, A′=CH₃CH₂;
Compound 5: R═R′=oleic acid group, n=1, A=CH₃, A′=CH₃;
Compound 6: R═R′=oleic acid group, n=2, A=CH₃, A′=CH₃;
Compound 7: R═R′=oleic acid group, n=3, A=CH₃, A′=CH₃;
Compound 8: R═R′=oleic acid group, n=4, A=CH₃, A′=CH₃;
Compound 9: R═R′=linoleic acid group, n=1, A=CH₃CH₂, A′=CH₃CH₂;
Compound 10: R═R′=linoleic acid group, n=2, A=CH₃CH₂, A′=CH₃CH₂;
Compound 11: R═R′=linoleic acid group, n=3, A=CH₃CH₂, A′=CH₃CH₂;
Compound 12: R═R′=linoleic acid group, n=4, A=CH₃CH₂, A′=CH₃CH₂;
Compound 13: R═R′=linoleic acid group, n=1, A=CH₃CH₂, A′=CH₃;
Compound 14: R═R′=linoleic acid group, n=2, A=CH₃CH₂, A′=CH₃;
Compound 15: R═R′=linoleic acid group, n=3, A=CH₃CH₂, A′=CH₃;
Compound 16: R═R′=linoleic acid group, n=4, A=CH₃CH₂, A′=CH₃;
Compound 17: R═R′=oleic acid group, n=1, A=CH₃CH₂, A′=CH₃CH₂;
Compound 18: R═R′=oleic acid group, n=2, A=CH₃CH₂, A′=CH₃CH₂;
Compound 19: R═R′=oleic acid group, n=3, A=CH₃CH₂, A′=CH₃CH₂;
Compound 20: R═R′=oleic acid group, n=4, A=CH₃CH₂, A′=CH₃CH₂;
Compound 21: R═R′=oleic acid group, n=1, A=CH₃, A′=CH₃CH₂;
Compound 22: R═R′=oleic acid group, n=2, A=CH₃, A′=CH₃CH₂;
Compound 23: R═R′=oleic acid group, n=3, A=CH₃, A′=CH₃CH₂;
Compound 24: R═R′=oleic acid group, n=4, A=CH₃, A′=CH₃CH₂.

In the embodiments as described herein, the compounds having Formula II are selected from group consisting of following compounds:
Compound 1′: R═R′=linoleic acid group, n=1, A=CH₃, A′=CH₃CH₂;
Compound 2′: R═R′=linoleic acid group, n=2, A=CH₃, A′=CH₃CH₂;
Compound 3′: R═R′=linoleic acid group, n=3, A=CH₃, A′=CH₃CH₂;
Compound 4′: R═R′=linoleic acid group, n=4, A=CH₃, A′=CH₃CH₂;
Compound 5′: R═R′=oleic acid group, n=1, A=CH₃, A′=CH₃;
Compound 6′: R═R′=oleic acid group, n=2, A=CH₃, A′=CH₃;
Compound 7′: R═R′=oleic acid group, n=3, A=CH₃, A′=CH₃;
Compound 8′: R═R′=oleic acid group, n=4, A=CH₃, A′=CH₃;
Compound 9′: R═R′=linoleic acid group, n=1, A=CH₃CH₂, A′=CH₃CH₂;
Compound 10′: R═R′=linoleic acid group, n=2, A=CH₃CH₂, A′=CH₃CH₂;
Compound 11′: R═R′=linoleic acid group, n=3, A=CH₃CH₂, A′=CH₃CH₂;
Compound 12′: R═R′=linoleic acid group, n=4, A=CH₃CH₂, A′=CH₃CH₂;
Compound 13′: R═R′=linoleic acid group, n=1, A=CH₃CH₂, A′=CH₃;
Compound 14′: R═R′=linoleic acid group, n=2, A=CH₃CH₂, A′=CH₃;
Compound 15′: R═R′=linoleic acid group, n=3, A=CH₃CH₂, A′=CH₃;
Compound 16′: R═R′=linoleic acid group, n=4, A=CH₃CH₂, A′=CH₃;
Compound 13′: R═R′=oleic acid group, n=1, A=CH₃CH₂, A′=CH₃CH₂;
Compound 14′: R═R′=oleic acid group, n=2, A=CH₃CH₂, A′=CH₃CH₂;
Compound 15′: R═R′=oleic acid group, n=3, A=CH₃CH₂, A′=CH₃CH₂;
Compound 16′: R═R′=oleic acid group, n=4, A=CH₃CH₂, A′=CH₃CH₂;
Compound 17′: R═R′=oleic acid group, n=1, A=CH₃, A′=CH₃CH₂;
Compound 18′: R═R′=oleic acid group, n=2, A=CH₃, A′=CH₃CH₂;
Compound 19′: R═R′=oleic acid group, n=3, A=CH₃, A′=CH₃CH₂;
Compound 20′: R═R′=oleic acid group, n=4, A=CH₃, A′=CH₃CH₂.

In the embodiments as described herein, the compounds having Formula III are selected from group consisting of following compounds:
Compound 1″: R═R′=linoleic acid group, n=1, A=CH₃, A′=CH₃CH₂;
Compound 2″: R═R′=linoleic acid group, n=2, A=CH₃, A′=CH₃CH₂;
Compound 3″: R═R′=linoleic acid group, n=3, A=CH₃, A′=CH₃CH₂;
Compound 4″: R═R′=linoleic acid group, n=4, A=CH₃, A′=CH₃CH₂;
Compound 5″: R═R′=oleic acid group, n=1, A=CH₃, A′=CH₃;
Compound 6″: R═R′=oleic acid group, n=2, A=CH₃, A′=CH₃;
Compound 7″: R═R′=oleic acid group, n=3, A=CH₃, A′=CH₃;
Compound 8″: R═R′=oleic acid group, n=4, A=CH₃, A′=CH₃;
Compound 9″: R═R′=linoleic acid group, n=1, A=CH₃CH₂, A′=CH₃CH₂;
Compound 10″: R═R′=linoleic acid group, n=2, A=CH₃CH₂, A′=CH₃CH₂;
Compound 11″: R═R′=linoleic acid group, n=3, A=CH₃CH₂, A′=CH₃CH₂;
Compound 12″: R═R′=linoleic acid group, n=4, A=CH₃CH₂, A′=CH₃CH₂;
Compound 13″: R═R′=linoleic acid group, n=1, A=CH₃CH₂, A′=CH₃;
Compound 14″: R═R′=linoleic acid group, n=2, A=CH₃CH₂, A′=CH₃;
Compound 15″: R═R′=linoleic acid group, n=3, A=CH₃CH₂, A′=CH₃;
Compound 16″: R═R′=linoleic acid group, n=4, A=CH₃CH₂, A′=CH₃;
Compound 17″: R═R′=oleic acid group, n=1, A=CH₃CH₂, A′=CH₃CH₂;
Compound 18″: R═R′=oleic acid group, n=2, A=CH₃CH₂, A′=CH₃CH₂;
Compound 19″: R═R′=oleic acid group, n=3, A=CH₃CH₂, A′=CH₃CH₂;
Compound 20″: R═R′=oleic acid group, n=4, A=CH₃CH₂, A′=CH₃CH₂;
Compound 21″: R═R′=oleic acid group, n=1, A=CH₃, A′=CH₃CH₂;
Compound 22″: R═R′=oleic acid group, n=2, A=CH₃, A′=CH₃CH₂;
Compound 23″: R═R′=oleic acid group, n=3, A=CH₃, A′=CH₃CH₂;
Compound 24″: R═R′=oleic acid group, n=4, A=CH₃, A′=CH₃CH₂.

In the embodiments as described herein, the compounds having Formula IV are selected from group consisting of following compounds:
Compound 1″′: R═R′=linoleic acid group, n=1, A=CH₃, A′=CH₃CH₂;
Compound 2″′: R═R′=linoleic acid group, n=2, A=CH₃, A′=CH₃CH₂;
Compound 3″′: R═R′=linoleic acid group, n=3, A=CH₃, A′=CH₃CH₂;
Compound 4″′: R═R′=linoleic acid group, n=4, A=CH₃, A′=CH₃CH₂;
Compound 5″′: R═R′=oleic acid group, n=1, A=CH₃, A′=CH₃;
Compound 6″′: R═R′=oleic acid group, n=2, A=CH₃, A′=CH₃;
Compound 7″′: R═R′=oleic acid group, n=3, A=CH₃, A′=CH₃;
Compound 8″′: R═R′=oleic acid group, n=4, A=CH₃, A′=CH₃;
Compound 9″′: R═R′=linoleic acid group, n=1, A=CH₃CH₂, A′=CH₃CH₂;
Compound 10″′: R═R′=linoleic acid group, n=2, A=CH₃CH₂, A′=CH₃CH₂;
Compound 11″′: R═R′=linoleic acid group, n=3, A=CH₃CH₂, A′=CH₃CH₂;
Compound 12″′: R═R′=linoleic acid group, n=4, A=CH₃CH₂, A′=CH₃CH₂;
Compound 13″′: R═R′=linoleic acid group, n=1, A=CH₃CH₂, A′=CH₃;
Compound 14″′: R═R′=linoleic acid group, n=2, A=CH₃CH₂, A′=CH₃;
Compound 15″′: R═R′=linoleic acid group, n=3, A=CH₃CH₂, A′=CH₃;
Compound 16″′: R═R′=linoleic acid group, n=4, A=CH₃CH₂, A′=CH₃;
Compound 17″′: R═R′=oleic acid group, n=1, A=CH₃CH₂, A′=CH₃CH₂;
Compound 18″′: R═R′=oleic acid group, n=2, A=CH₃CH₂, A′=CH₃CH₂;
Compound 19″′: R═R′=oleic acid group, n=3, A=CH₃CH₂, A′=CH₃CH₂;
Compound 20″′: R═R′=oleic acid group, n=4, A=CH₃CH₂, A′=CH₃CH₂;
Compound 21′″: R═R′=oleic acid group, n=1, A=CH₃, A′=CH₃CH₂;
Compound 22″′: R═R′=oleic acid group, n=2, A=CH₃, A′=CH₃CH₂;
Compound 23″′: R═R′=oleic acid group, n=3, A=CH₃, A′=CH₃CH₂;
Compound 24″′: R═R′=oleic acid group, n=3, A=CH₃, A′=CH₃CH₂.

The cholesterol lipid compound as described herein has a chemical structure represented by Formula V:

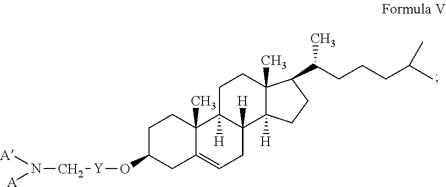

wherein, Y═(C═O), (C═S), (—HN(O)C—) or bond, A and A′ is C₁-C₄ alkyl, n=1-4ₒ

In the embodiments as described herein, the compounds having Formula V are selected from group consisting of following compounds:
Compound V1: Y═(C═O), n=1, A=CH₃, A′=CH₃CH₂;
Compound V2: Y═(C═O), n=2, A=CH₃, A′=CH₃CH₂;
Compound V3: Y═(C═O), n=3, A=CH₃CH₂, A′=CH₃CH₂;
Compound V4: Y═(C═O), n=4, A=CH₃CH₂, A′=CH₃CH₂;
Compound V5: Y═(C═O), n=1, A=CH₃CH₂, A′=CH₃;
Compound V6: Y═(C═O), n=2, A=CH₃, A′=CH₃;
Compound V7: Y═(C═O), n=3, A=CH₃, A′=CH₃;
Compound V8: Y═(C═O), n=4, A=CH₃, A′=CH₃;
Compound V9: Y═(C═S), n=1, A=CH₃CH₂, A′=CH₃CH₂;
Compound V10: Y═(C═S), n=2, A=CH₃CH₂, A′=CH₃CH₂;
Compound V11: Y═(C═S), n=3, A=CH₃CH₂, A′=CH₃CH₂;
Compound V12: Y═(C═S), n=4, A=CH₃CH₂, A′=CH₃CH₂;
Compound V13: Y═(C═S), n=1, A=CH₃CH₂, A′=CH₃;
Compound V14: Y═(C═S), n=2, A=CH₃CH₂, A′=CH₃;
Compound V15: Y═(C═S), n=3, A=CH₃CH₂, A′=CH₃;
Compound V16: Y═(C═S), n=4, A=CH₃CH₂, A′=CH₃;
Compound V17: Y═(—HNC═O—), n=1, A=CH₃CH₂, A′=CH₃CH₂;
Compound V18: Y═(—HNC═O—), n=2, A=CH₃CH₂, A′=CH₃CH₂;
Compound V19: Y═(—HNC═O—), n=3, A=CH₃CH₂, A′=CH₃CH₂;
Compound V20: Y═(—HNC═O—), n=4, A=CH₃CH₂, A′=CH₃CH₂;
Compound V21: Y═(—HNC═O—), n=1, A=CH₃, A′=CH₃CH₂;
Compound V22: Y═(—HNC═O—), n=2, A=CH₃, A′=CH₃CH₂;
Compound V23: Y═(—HNC═O—), n=3, A=CH₃, A′=CH₃CH₂;
Compound V24: Y═(—HNC═O—), n=4, A=CH₃, A′=CH₃CH₂.

In another aspect, a nucleic acid liposome formulation is provided herein, comprising the liposome as described above and a nucleic acid encapsulated by the liposome. The nucleic acid is selected from the group consisting of small interfering nucleic acid, micro nucleic acid, non-coding nucleic acid, antisense nucleic acid, small ligand nucleic acid and small active nucleic acid. The nucleic acid liposome formulation as described herein may further comprise a pharmaceutically acceptable excipient. In one embodiment, the nucleic acid liposome formulation as described herein may be in the form of tablet, capsule, lotion, drop, power, solution or aerosol. In another embodiment, the nucleic acid liposome formulation as described herein may be selected from the group consisting of an oral formulation, an intravascular injection formulation, an intramuscular injection formulation, a subcutaneous administration formulation, a parentenral administration formulation, and an intraperitoneal administration formulation.

In yet another aspect, a method for preparing the nucleic acid liposome formulation as mentioned above is provided herein, including:
(1) providing a mixture solution of the cationic lipid compound, the phospholipid and the PEGylated lipid;
(2) mixing the said mixture solution and the nucleic acid solution to obtain the nucleic acid liposome formulation.

In the mixture solution, the cationic lipid compound is in an amount from 50 wt % to 90 wt %.

Preferably, after step (1), the mixture solution is subject to extrusion process to obtain empty liposome vesicles. The nucleic acid solution is mixed with the empty liposome vesicles, such that the nucleic acid is loaded into the empty liposome vesicles to form nucleic acid liposome formulation.

In still yet another aspect, a method for treating diseases associated with abnormal expression of genes is provided herein, including administering the nucleic acid liposome formulation as described herein to a subject in need of such treatment. The nucleic acid liposome formulation as described herein is orally, intravascularly, intramuscularly, subcutaneously, parenternrally or intraperitoneally administered to the patient. In some embodiments as described herein, the nucleic acid encapsulated by the nucleic acid liposome formulation can be delivered into cells, such as cells of target tissues such as liver, inflammation tissues and the like.

The present invention also relates to use of the nucleic acid liposome formulation as described herein in manufacturing a medicament for treating diseases associated with abnormal expression of genes.

The advantages achieved by the present invention comprises:
1) Increased loading capacity. Since the liposome as provided herein comprises the cationic lipid compound in high amount, the nucleic acid with negative charges can be loaded into the liposome in increased amount, such that the loading capacity for the nucleic acid is greatly increased.
2) Reduced drug toxicity. Since increased nucleic acid drugs can be loaded into the unit lipid vesicles of the liposome formulation, in the case of administration of drug at equivalent dosage, intake of the cationic lipid compound is relatively decreased due to increased loading capacity, thereby leading to reduced toxicity.

### BRIEF DESCRIPTION OF THE DRAWINGS

FIG. 1 is a column diagram of mice body weights before and after administering the liposome formulation with the nucleic acid drug encapsulated therein according to one embodiment as described herein, wherein, the horizontal axis shows respective experiment groups, and the vertical axis shows mice body weights.

FIG. 2 is a column diagram of relative mRNA expression level of ApoB gene in mice's liver before and after administering the liposome formulation with the nucleic acid drug encapsulated therein to the mice according to one embodiment as described herein, wherein, the horizontal axis shows respective experiment groups, and the vertical axis shows relative mRNA expression level of ApoB gene.

FIG. 3 is a column diagram of total cholesterol level in mice's serum before and after administering the liposome formulation with the nucleic acid drug encapsulated therein to the mice according to one embodiment as described herein, wherein, the horizontal axis shows respective experiment groups, and the vertical axis shows total cholesterol level in mice's serum.

FIG. 4 is a column diagram of mice body weights before and after administering the liposome formulation with the nucleic acid drug encapsulated therein to the mice according to another embodiment as described herein, wherein, the horizontal axis shows respective experiment groups, and the vertical axis shows mice body weights.

FIG. 5 is a column diagram of relative mRNA expression level of ApoB gene in mice's liver before and after administering the liposome formulation with the nucleic acid drug encapsulated therein to the mice according to another embodiment as described herein, wherein, the horizontal axis shows respective experiment groups, and the vertical axis shows relative mRNA expression level of ApoB gene.

FIG. 6 is a column diagram of total cholesterol level in mice's serum before and after administering the liposome formulation with the nucleic acid drug encapsulated therein to the mice according to another embodiment as described herein, wherein, the horizontal axis shows respective experiment groups, and the vertical axis shows total cholesterol level in mice's serum.

FIG. 7 is a column diagram of mice body weights before and after administering the liposome formulation with the nucleic acid drug encapsulated therein to the mice according to yet another embodiment as described herein, wherein, the horizontal axis shows respective experiment groups, and the vertical axis shows mice body weights.

FIG. 8 is a column diagram of relative mRNA expression level of ApoB gene in mice's liver before and after administering the liposome formulation with the nucleic acid drug encapsulated therein to the mice according to yet another embodiment as described herein, wherein, the horizontal axis shows respective experiment groups, and the vertical axis shows relative mRNA expression level of ApoB gene.

FIG. 9 is a column diagram of total cholesterol level in mice's serum before and after administering the liposome formulation with the nucleic acid drug encapsulated therein to the mice according to yet another embodiment as described herein, wherein, the horizontal axis shows respective experiment groups, and the vertical axis shows total cholesterol level in mice's serum.

FIG. 10 is a column diagram of mice body weights before and after administering the liposome formulation with the nucleic acid drug encapsulated therein to the mice according to still yet another embodiment as described herein, wherein, the horizontal axis shows respective experiment groups, and the vertical axis shows mice body weights.

FIG. 11 is a column diagram of relative mRNA expression level of ApoB gene in mice's liver before and after administering the liposome formulation with the nucleic acid drug encapsulated therein to the mice according to still yet another embodiment as described herein, wherein, the horizontal axis shows respective experiment groups, and the vertical axis shows relative mRNA expression level of ApoB gene.

FIG. 12 is a column diagram of total cholesterol level in mice's serum before and after administering the liposome formulation with the nucleic acid drug encapsulated therein to the mice according to still yet another embodiment as described herein, wherein, the horizontal axis shows respective experiment groups, and the vertical axis shows total cholesterol level in mice's serum.

FIG. 13 is a column diagram of mice body weights before and after administering the liposome formulation with the nucleic acid drug encapsulated therein to the mice according to still yet another embodiment as described herein, wherein, the horizontal axis shows respective experiment groups, and the vertical axis shows mice body weights.

FIG. 14 is a column diagram of relative mRNA expression level of ApoB gene in mice's liver before and after administering the liposome formulation with the nucleic acid drug encapsulated therein to the mice according to still yet another embodiment as described herein, wherein, the horizontal axis shows respective experiment groups, and the vertical axis shows relative mRNA expression level of ApoB gene.

FIG. 15 is a column diagram of total cholesterol level in mice's serum before and after administering the liposome formulation with the nucleic acid drug encapsulated therein to the mice according to still yet another embodiment as described herein, wherein, the horizontal axis shows respective experiment groups, and the vertical axis shows total cholesterol level in mice's serum.

FIG. 16 is a column diagram of mice body weights before and after administering the liposome formulation with the nucleic acid drug encapsulated therein to the mice according to still yet another embodiment as described herein, wherein, the horizontal axis shows respective experiment groups, and the vertical axis shows mice body weights.

FIG. 17 is a column diagram of relative mRNA expression level of ApoB gene in mice's liver before and after administering the liposome formulation with the nucleic acid drug encapsulated therein to the mice according to still yet another embodiment as described herein, wherein, the horizontal axis shows respective experiment groups, and the vertical axis shows relative mRNA expression level of ApoB gene.

FIG. 18 is a column diagram of total cholesterol level in mice's serum before and after administering the liposome formulation with the nucleic acid drug encapsulated therein to the mice according to still yet another embodiment as described herein, wherein, the horizontal axis shows respective experiment groups, and the vertical axis shows total cholesterol level in mice's serum.

### DETAILED DESCRIPTION OF THE INVENTION

Several aspects of the invention are described below in details by reference to appended drawing and specific embodiments. The skilled in the art should understand that the embodiments are set forth to provide an illustration, rather than limit the scope of the present invention. The scope of the present invention is limited by the appended claims.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

### EXAMPLE 1

The lipid-type lipid compound and the cholesterol lipid compound used in the liposome formulation as described herein were prepared according to the relevant reference documents, patents and patent applications or modified technologies in organic chemistry, synthetic chemistry and biological chemistry fields. For example, the lipid-type lipid compound (CL52) having Formula I wherein R═R′=linoleic acid group, n=1, A=A′=CH₃ was prepared according to the methods that can be found in Heyes J et. al., J Control Release 2005; 107:276-287. The lipid-type lipid compound (CL53) having Formula I wherein R═R′=oleic acid group, n=1, A=A′=CH₃ was prepared according to the methods that can be found in Heyes J et. al., J Control Release 2005; 107:276-287. The lipid-type lipid compound (CL54) having Formula II wherein R═R′=linoleic acid group, n=2, A=A′=CH₃ was prepared according to the methods that can be found in J Chen et al., PCT applications WO/2009/086558 and WO/2010/042877 or Semple S C et al., Nature Biotech, 2010; 28:172-176. The lipid-type lipid compound (CL51) having Formula III wherein R═R′=linoleic acid group, n=3, A=A′=CH₃ was prepared according to the methods that can be found in Jayaraman M et al., Angew Chem Int Ed Engl. 2012; 51:8529-33. The lipid-type lipid compound (CL55) having Formula IV wherein R═R′=linoleic acid group, n=1, A=A′=CH₃ was prepared according to the methods that can be found in Leventis R et al., Biochim Biophys Acta 1990; 1023:124-132. The lipid-type lipid compound (CL56) having Formula IV wherein R═R′=oleic acid group, n=1, A=A′=CH₃ was prepared according to the methods that can be found in Leventis R et al., Biochim Biophys Acta 1990; 1023:124-132. PEG-c-DMA was prepared according to the methods that can be found in J Chen et al., PCT applications WO/2009/086558 and WO/2010/042877 or Semple S C et al., Nature Biotech, 2010; 28:172-176.

The cholesterol lipid compound (CL66) having Formula V wherein Y═(C═O), A=A′=CH₃, n=2 was prepared as follows.

Cholesterol (1.1 g, 2.84 mmol) dissolved in DCM (20 mL) and material 11 (4-(dimethylamino)propionic acid hydrochloride, 0.7 g, 4.55 mmol) were added into 100 mL flask and then di-isopropyl ethylamine (1 mL) and DMAP (0.056 g, 0.56 mmol) were added thereto. The mixture in the flask was stirred for 5 min at room temperature and EDC.HCl (1.1 g, 5.6 mmol) was added and stirred overnight at room temperature. Progresses of reaction was detected by TLC (5% MeOH/DCM). After completing the reaction, 20 mL DCM was added to dilute the solution. 16 mL saturated NaHCO₃, 16 mL water and 16 mL saturated saline were used to wash the solution. The organic phase was dried over anhydrous Na₂SO₄, filtered and concentrated, to yield 1.1 g crude product. The crude product was purified by using Flash column (90 g silica gel, loading by 210 mL DCM containing 0.1% TEA; the moving phases were respectively: 140 mL DCM containing 0.1% TEA, 560 ml 2% methanol+98% DCM containing 0.1% TEA; 140 mL 2.5% methanol+97.5% DCM containing 0.1% TEA, 420 mL 3% methanol +97% DCM containing 0.1 TEA) to yield purified product CL66.

The cholesterol lipid compound (CL68) having Formula V wherein Y═(C═O), A=A′=CH₃, n=1 was prepared as follows.

Cholesterol (1.1 g, 2.84 mmol) dissolved in DCM (20 mL) and material 13 (4-(dimethylamino)propionic acid hydrochloride, 0.7 g, 5.0 mmol) were added into 100 mL flask and then di-isopropyl ethylamine (1 mL) and DMAP (0.056 g, 0.56 mmol) were added thereto. The mixture in the flask was stirred for 5 min at room temperature and EDC.HCl (1.1 g, 5.6 mmol) was added and stirred overnight at room temperature. Progresses of reaction were detected by TLC (5% MeOH/DCM). After completing the reaction, 20 mL DCM was added to dilute the solution. 16 mL saturated NaHCO₃, 16 mL water and 16 mL saturated saline were used to wash the solution. The organic phase was dried over anhydrous Na₂SO₄, filtered and concentrated, to yield 1.1 g crude product. The crude product was purified by using Flash column (90 g silica gel, loading by 210 mL DCM containing 0.1% TEA; the moving phases were repectively: 140 mL DCM containing 0.1% TEA, 560 ml 2% methanol +98% DCM with containing 0.1% TEA; 140 mL 2.5% methanol+97.5% DCM containing 0.1% TEA, 420 mL 3% methanol+97% DCM containing 0.1% TEA) to yield purified product CL68.

### EXAMPLE 2

Preparation of Liposome Formulation

1. Reagents, Materials and Instruments

1.1 Reagents:

The lipid-type lipid compound was compound CL51 having Formula III wherein R═R′=linoleic acid group and n=3.

The cholesterol lipid compounds were compounds CL66 and CL68 prepared according to Example 1.

The phospholipid was distearoyl phosphatidylcholine (DSPC) purchased from Shanghai Advanced Vehicle Technology L.T.D. Co.

The PEGylated lipid was PEG-c-DMA prepared according to Example 1.

The fluorescent dye, potassium 6-(p-toluidino)-2-naphthalenesulphonate (TNS), was purchased from Sigma-Aldrich Co.

Other reagents including absolute ethanol, methanol, chloroform, citric acid, sodium citrate, NaCl and so on were imported or domestic analytical grade reagents.

The exemplary nucleic acid medicament used in the present example was ApoB-siRNA synthesized by BIOMICS BIOTECHNOLOGIES CO., LTD. in the form of freeze-dried powder, with following sequence:

ApB-S: 5′-GUCAUCACACUGAAUACCAAU-3′;
ApB-AS: 5′-AUUGGUAUUCAGUGUGAUGACAC-3′.

1.2 Materials: ion exchanging column: Vivapure D Mini H (Sartorius stedim Co., USA), Ultracel-100 centrifugal ultrafiltration tube (Millipore Co., USA), dialysis membrane: Nuclepore Membrane Circles (80 nm) (Whatman Co., USA), dialysis bag and needle filter purchased from Sangon Biotech Co. Ltd., dialysis bag MD24 (MW: 20000, Shanghai Baoman Biotechnology Co. Ltd.), needle filter (Sangon Biotech Co. Ltd.), 50 mL Falcon tube (Corning Co., USA).

1.3 Instruments: Vortex Mixer (VWR Co., USA), constant temperature water bath (Jiangsu Tianhong Instrument Co.), Centrifuge (Eppendorf Co., USA), UV-Vis Spectrophotometer (Shanghai Jingke Co.), Magnetic Stirrer (Shanghai Meiyingpu Instrument Co.), NLI Liposome Extruder (ATS Co., CA), Clean Bench (Suzhou Jinghua), High Speed Refrigerated Centrifuge (Saite Xiangyi), Fluorospectro Photometer (RF-5301PC, Shimadzu Co., JP) and so on.

2. Preparation of Liposome Formulation

2.1 Preparation of Reagents:

2.1.1 Preparation of Lipid Stock Solution

The lipid-type lipid compound CL51, the cholesterol lipid compounds CL66 and CL68, the phospholipid DSPC, and the PEGylated lipid PEG-c-DMA were balanced at room temperature for about 30 min before weighting.

The lipid-type lipid compound CL51 was formulated to 40 mg/mL stock solution by using 100% ethanol. The cholesterol lipid compounds CL66 and CL68 were formulated to 20 mg/mL stock solution by using 100% ethanol, respectively. DSPC was formulated to 20 mg/mL stock solution by using 100% ethanol. The PEGylated lipid PEG-c-DMA was formulated to 50 mg/mL stock solution by using 100% ethanol.

2.2 Preparation of Liposome Formulations

The liposome formulations F61-F520 were prepared at the ratio of the lipid-type lipid compound CL51 to the cholesterol lipid compound CL68 as listed in Table 1 (wherein, the PEGylated lipid was PEG-c-DMA at amount of 10% and the remainder was DSPC to arrive at 100%, and the amounts of the PEGylated lipid and the phospholipid as mentioned above were used in following examples). In particular, the lipid-type lipid compound CL51, the cholesterol lipid compound CL68, the PEGylated lipid PEG-c-DMA and phospholipid DSPC at the ratios as listed in table 1 were formulated into ethanol solution by using absolute ethanol as solvent. After all of the lipids were dissolved, the ethanol solution of the lipids as mentioned above was added into buffer solution (50 mM citric acid, pH 4.0) with stirring, to obtain 30% (v/v) of final ethanol concentration. At room temperature, under pressure of 200 psi, extrusion was performed by using NLI liposome extruder equipped with two layers of Nuclepore Membrane Circles. Empty liposome vesicles with uniform diameter were obtained. The diameter of the empty liposome vesicles obtained through extruder was measured by particle size analyzer.

TABLE 1

| | Amount of | | | |
|---|---|---|---|---|
| | cationic | Lipid-type lipd | | |
| | lipid | compound:Cholesterol | | Cholesterol |
| Formu- | compound | lipid | Lipid | lipid |
| lation | (%) | compound | compound | compound |
| F601 | 60 | 4:1 | CL51 | CL68 |
| F602 | 60 | 2:2 | CL51 | CL68 |
| F603 | 60 | 1:4 | CL51 | CL68 |
| F608 | 60 | 2:1 | CL51 | CL68 |
| F609 | 60 | 2:1 | CL51 | CL66 |
| F610 | 60 | 1:1 | CL51 | CL66 |
| F504 | 70 | 4:1 | CL51 | CL68 |
| F505 | 75 | 4:1 | CL51 | CL68 |
| F506 | 80 | 4:1 | CL51 | CL66 |
| F507 | 80 | 2:2 | CL51 | CL68 |
| F510 | 80 | 1:4 | CL51 | CL68 |
| F511 | 83 | 4:1 | CL51 | CL68 |
| F512 | 85 | 4:1 | CL51 | CL68 |
| F513 | 88 | 4:1 | CL51 | CL68 |
| F518 | 90 | 4:1 | CL51 | CL68 |
| F519 | 90 | 2:2 | CL51 | CL66 |
| F520 | 90 | 1:4 | CL51 | CL66 |

2.3 Preparation of Liposome Formulation with siRNA Encapsulated Therein

2.3.1 Preparation of siRNA Stock Solution:

Dissolution of siRNA: ApoB-siRNA stock solution with 50 mg/mL of theorical final concentration was formulated by using solution of 10 mM citric acid/30 mM NaCl at pH of 6.0. ApoB-siRNA stock solution in a suitable amount was diluted 2000 times by PBS for detection of concentration of siRNA. Each sample was repeated in triplicate. A₂₆₀ value was detected by UV-Vis Spectrophotometer to calculate actual concentration of ApoB-siRNA stock solution.

The empty liposome vesicles at desired volume was added into 50 mL Falcon tube to prebalance in water bath at 35° C. for 4-5min. The ApoB-siRNA stock solution was formulated to siRNA/formulation buffer/ethanol solution to make sure that ethanol was comprised of 30% relative to total volume. The siRNA/formulation buffer/ethanol solution was slowly added to the empty liposome vesicles and incubated at 35° C. for 30min. After incubation, 600 μL incubated product was used for A₂₆₀ analysis. The siRNA liposome formulation was subject to dialysis overnight by using dialysis bag MD24 in 1× PBS. The dialyzed product was recovered and filtered by using a 0.2 μm needle filter to remove bacteria. After removing bacteria, 600 μL product was used for detection of encapsulation efficiency.

2.4 Detection of Encapsulation Efficiency

300 μL product from which bacteria was removed passes through Vivapure D Mini H (ion exchange column), to obtain post-chromatography sample. The remainder 300 μL product from which bacteria was removed was used as the sample without chromatography. The actual concentration coefficient (mg/mL) was calculated according to the actual A₂₆₀ value, i.e., the actual concentration coefficient (mg/mL)=volume of the added ApoB-siRNA stock solution (mL)×actual concentration of ApoB-siRNA stock solution (mg/mL)×0.03 (mL)×1000/(total volume of siRNA liposome formulation (mL)×A₂₆₀ value of incubated product×1.1 (mL)). The encapsulation efficiency was calculated according to siRNA concentration. The encapsulation efficiency can be defined as encapsulation efficiency=(the medicaments encapsulated in the liposome/total amount of the medicaments in the liposome formulation)×100%. The encapsulation efficiency can be calculated according to the equation: encapsulation efficiency=siRNA concentration of the post-chromatography sample/siRNA concentration of the sample without chromatography.

Loading efficiency=(the amount of the medicaments encapsulated in the liposome/liposome+total amount of the medicaments in the liposome formulation)×100%. 2.5

2.6 Detection of pKa of the Empty Liposome Vesicles

The fluorescent dye TNS radiated no luminescence or weak luminescence in aqueous solution, while it radiated strong luminescence in organic environment. pKa value of the empty liposome vesicles can be detected by taking advantage of fluorescent characteristics of TNS. When TNS with negative charges was added into the solution of cationic liposomes, it aggregated on the surfaces of the liposomes to radiate fluorescence; while free TNS in the aqueous solution radiated no luminescence. Therefore, the fluorescent density of the solution (within a certain concentration range) exhibited positive proportion to the amount of the positive charges on the surfaces of the liposomes. As such, ionization extent of amino in the cation liposomes can be obtained by detecting the fluorescent density. According to the definition of the dissociation constant, when the ionization extent of the weak acid in the solution arrived at 50%, the pH value of the solution was the pKa value of the weak acid. Therefore, according to the diagram of the fluorescent density relative to the pH value, pH value corresponding to the middle point of the fluorescent density was the apparent pKa value of the cation solution.

The particular detection method included:
1) adding 2 mL PBS solution to each sample flask and adjusting pH values of PBS solution in each sample flask to various pH values (2.5, 3.0, 3.5, 4.0, 4.5, 5.0, 5.5, 6.0, 6.5, 7.0, 7.5, 8.0, 8.5, 9.0, 9.5, 10.0, 10.5, 11.0 respectively) by HCl and NaOH;
2) adding to each sample flask 80 μL 250 mM 4-morpholine ethanesulfonic acid solution, 80 μL 250 mM HEPES solution, 80 μL 250 mM ammonium acetate solution, 80 μL 3.85 M sodium chloride solution and 60 μL PFV in turn;
3) adding 40 mL 83.5 μM TNS solution to the sample flask containing PBS solution at pH value of 2.5 and stirring for 30 s, then removing the solution and measuring the fluorescent density thereof by using fluorospectro photometer (excitation wavelength is 321 nm, emission wavelength is 445 nm);
4) according to the steps as mentioned above, measuring fluorescent density of TNS under various pH values, plotting a diagram with horizontal axis of pH value and vertical axis of fluorescent density, wherein pH value corresponding to the middle point of the fluorescent density is apparent pKa value of PFV, thereby obtaining pKa value.

2.7 Analysis of Particle Size

Particle sizes of the empty liposome vesicles and siRNA liposome formulations were measured by Nicomp 370 particle size analyser.

Table 2 listed the particle sizes, pKa values of the empty liposome vesicles prepared as mentioned above and encapsulation efficiencies and loading efficiencies of the empty liposome vesicles for siRNA.

TABLE 2

| | | | Encapsulation | Loading |
|---|---|---|---|---|
| | Particle Size | | Efficiency | Efficiencies |
| Formulation | (nm) | pKa Value | for siRNA | for siRNA |
| F601 | 63.35 | 5.77 | 65.9 | 68.9 |
| F602 | 62.01 | 6.01 | 66.1 | 67.3 |
| F603 | 65.09 | 5.93 | 63.4 | 69.4 |
| F608 | 63.15 | 6.55 | 67.9 | 65.2 |
| F609 | 69.31 | 7.32 | 55.4 | 68.3 |
| F610 | 68.13 | 7.45 | 36.6 | 69.0 |
| F504 | 69.35 | 6.31 | 71.5 | 70.3 |
| F505 | 66.29 | 7.32 | 69.3 | 78.7 |
| F506 | 65.49 | 7.51 | 65.5 | 76.3 |
| F507 | 68.75 | 6.55 | 69.7 | 77.9 |
| F510 | 69.33 | 6.54 | 70.9 | 78.6 |
| F511 | 65.29 | 5.98 | 65.3 | 79.8 |
| F512 | 69.54 | 6.11 | 68.4 | 80.8 |
| F513 | 78.11 | 6.87 | 67.9 | 81.3 |
| F518 | 79.21 | 6.73 | 70.2 | 87.5 |
| F519 | 81.32 | 5.99 | 69.5 | 85.1 |
| F520 | 80.10 | 6.31 | 71.3 | 88.0 |

From the measurements of encapsulation efficiencies and loading efficiencies for siRNA as listed in Table 2, it can be seen that the encapsulation efficiencies and loading efficiencies for siRNA were increased as increase of the content of cationic lipid compound in the liposome formulation. The nucleic acid with negative charges loaded into the liposome formulation was increased due to increase of the content of the cationic lipid compound. Therefore, loading amount for the nucleic acid medicaments can be greatly increased by using the liposome formulation as described herein. As a result, increased amount of nucleic acid medicaments can be loaded into the unit lipid vesicles of the liposome formulation as described herein. In the case of administration of medicaments at the same dosage, intake of the cationic lipid compound was decreased due to increased loading amount for the nucleic acid medicaments, so as to decrease use of lipid and toxicity.

### EXAMPLE 3

Efficiency of the Liposome Formulation

1. Reagents, Materials and Instruments

1.1 Reagents and Materials: liposome formulation (as prepared according to Example 2), sodium chloride injection solution (Shandong Kangning Pharmaceutical Co. Ltd.), RISO™ RNA extraction reagent (BIOMICS BIOTECHNOLOGIES CO., LTD), 1 mL sterilized syringe (Henan Shuguangjianshi Medical Device Co. Ltd.), total cholesterol (CHO enzyme test) detection kit (Nanjing Jiancheng Biology Co.).

1.2 Experiment Animals: 4-6 weeks female ICR mice, 18-22 g, purchased from Comparison Medical Center of Nantong University.

1.3 Instruments: LightCycler 480 Real-time fluorescence quantification PCR analyser (Roche Co., USA), UV-Vis Spectrophotometer UV759 (Shanghai Jingke Co.).

2. Experimental Method

2.1 Experimental Grouping: weighting mice body weight before administration and grouping the mice according to the body weight into 3 groups: siRNA liposome formulation treated group (Table 1), saline treated group.
siRNA liposome formulation treated group: the injection amount was calculated according to 3 mg/kg of siRNA in the liposome;
saline treated group: 300 μL sodium chloride injection solution.

2.2 Administration: fixing mice by fixer and injecting liposome formulation in an amount of 3 mg/kg siRNA in the liposome formulation via tail vein of mice using 1 mL sterilized syringe; injecting 300 μL sodium chloride injection solution for control group.

2.3 Testing of Total Cholesterol Content in Serum Of Mice

Mice body weight was weighted at 48h upon administration. About 800 μL of blood of mice was collected by removing eyes and placed at 4° C. for 1 h. Serum was separated by centrifugation at 3000 rpm for 10 min for test. Total cholesterol in 10 μL separated mice serum was detected according to the instruction of the total cholesterol (CHO enzyme test) detection kit and absorbancy at 500 nm was detected.

Cholesterol content was calculated according to the equation that cholesterol content=(A₅₀₀ value of the testing sample/A₅₀₀ value of the standard sample) x concentration of standard solution (200 mg/dL (5.17 mM)).

2.4 Real-time quantification PCR (RT-qPCR) was used to detect mRNA expression level of ApoB gene in mice liver.

At 48 h upon injection of liposome formulation, mice were sacrificed and three blocks of tissues from various positions in liver were removed from mice. Total RNA of mice liver was extracted by using RISO™ RNA extraction reagent according to the manufacture's instruction. mRNA expression level of ApoB gene in mice liver was detected by RT-qPCR.

mRNA expression level of ApoB gene in the sample was detected by using primer with gene specificity and housekeeping gene GAPDH was amplified for internal reference. ApoB gene and internal reference gene GAPDH for each sample were amplified at the same time in triplicate. OneStep qPCR kit was used for quantification of reaction. The following reaction system was established: 2 μL RNA template, 12.5 μL 2× Master Mix, 0.5 μL for each 5′-end primer (10 μM) and 3′-end primer (10 μM), 0.5 μL 50× SYBR Green I Solution. The volume of the system was supplemented to 25 μL by using water without RNase. After mixing, the system was placed on the quantification PCR analyser for reaction.

The sequence of 5′-end primer for detection of mRNA of ApoB gene was AAGCACCTCCGAAAGTACGTG, and the sequence of 3′-end primer for detection of mRNA of ApoB gene is CTCCAGCTCTACCTTACAGTTGA. The sequence of 5′-end primer for detection of the housekeeping gene GAPDH was GTATGACTCCACTCACGGCAAA and the sequence of 3′-end primer for detection of the housekeeping gene GAPDH was GGTCTCGCTCCTGGAAGATG. All of the primers were synthesized by BIOMICS BIOTECHNOLOGIES CO., LTD.

Reaction condition comprises: reverse transcription for 30 min at 42° C., initial denaturation for 5 min at 95° C., denaturation for 20 sec at 95° C., anneal for 30 sec at 58° C., extension for 30 sec at 72° C., circulation for 45 times. Dissolution curve reaction is performed under95° C./5 min, 58° C./5 min and the temperature is increased to 95° C. at 0.5° C./5 sec.

2.5 Statistical Analysis

SPSS 14.0 statistical software was used and the data was shown as x̅±s. Significant difference among groups was detected by single factor variance analysis. Comparison between two groups was performed by t test. P value<0.05 was considered to be statistically significant.

3. Experimental Results

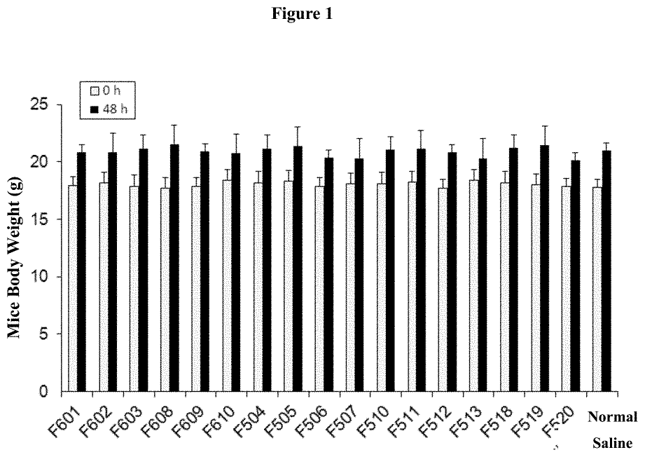

3.1 Variation of mice body weight. Variation of mice body weight before and after injection of liposome formulation was detected to indirectly reflect toxicity. As shown in FIG. 1, no significant variation of mice body weight was shown in each group before and after administration, indicating that the liposome formulation as described herein had no significant toxicity.

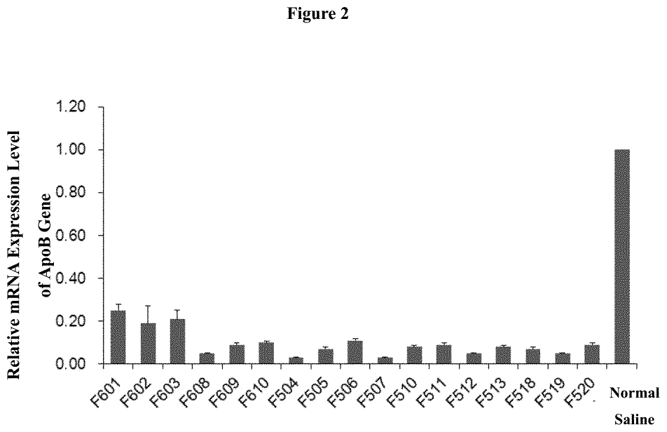

3.2 Variation of mRNA level of ApoB gene. As shown in FIG. 2, the liposome formation as described herein effectively inhibited expression of ApoB mRNA in liver.

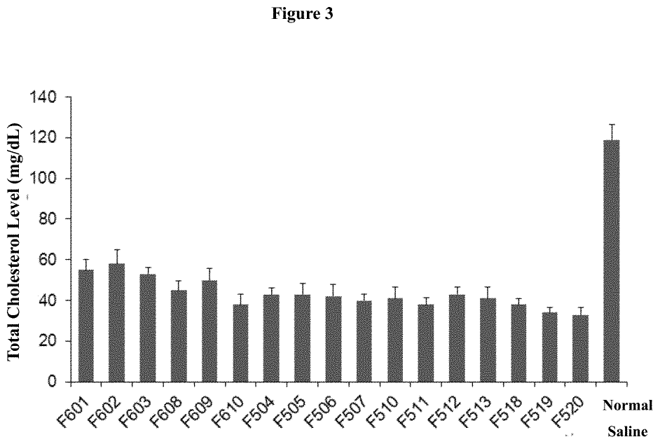

3.3 Variation of total cholesterol content. As shown in FIG. 3, in comparison with the control group, the total cholesterol level in serum of mice was decreased by using the liposome formulation as described herein.

In sum, it can be seen that the liposome formulation of the present invention was able to deliver nucleic acid medicaments such as siRNA to target tissues or organs so as to facilitate the nucleic acid medicaments to play their role.

### EXAMPLE 4

1. Preparation of Liposome Formulation with siRNAs Encapsulated Therein

1.1 The cationic lipid compound consisted of lipid compound CL52 and cholesterol lipid compound CL68. Phospholipid was DSPC purchased from Shanghai Shanghai Advanced Vehicle Technology L.T.D. Co. The PEGylated lipid was PEG-c-DMA.

1.2 The liposome formulation with siRNAs encapsulated therein was prepared according to the steps as described in Example 2 and parameters were detected. The ratios of the lipid-type lipid compound to the cholesterol lipid compound were listed in Table 3. Table 4 listed particle sizes and pKa values of the liposome formulations and the encapsulation efficiencies and loading efficiencies of the liposome formulations with siRNA encapsulated therein.

TABLE 3

| | Content | | | |
|---|---|---|---|---|
| | of cationic | Lipid-type lipd | | |
| | lipid | compound:Cholesterol | | Cholesterol |
| Formu- | compound | lipid | Lipid | lipid |
| lations | (%) | compound | compound | compound |
| F611 | 40 | 2:1 | CL52 | CL68 |
| F612 | 60 | 2:1 | CL52 | CL68 |
| F613 | 80 | 2:1 | CL52 | CL68 |
| F614 | 90 | 2:1 | CL52 | CL68 |

TABLE 4

| | | | siRNA | siRNA |
|---|---|---|---|---|
| | | | Encapsulation | Loading |
| Formulations | Particle Size (nm) | pKa value | Efficiency | Efficiency |
| F611 | 88.30 | 6.52 | 66.3 | 48.1 |
| F612 | 85.49 | 6.87 | 60.8 | 67.3 |
| F613 | 79.63 | 6.51 | 79.3 | 79.5 |
| F614 | 69.97 | 6.79 | 75.6 | 87.9 |

From the above table 4, it can be seen that siRNA encapsulation efficiency and loading efficiency were increased with increase of the content of cationic lipid compound in the liposome formulation. This indicated that the nucleic acid with negative charges loaded into the liposome was increased due to the increase of the the content of cationic lipid compound. Therefore, the loading efficiency for nucleic acid medicaments was greatly increased by using the liposome formulation as described herein. Therefore, increased nucleic acid medicament was loaded into the unit lipid vesicles of the liposome formulation as described herein. At the same dosage, intake of cationic lipid compound was decreased due to increased loading amount, thereby reducing use of the lipid and toxicity.

2. Efficiency of the Liposome Formulation with siRNAs Encapsulated Therein

The efficiency of the liposome formulation with siRNAs encapsulated therein as prepared in this example was detected according to the method as described in Example 3.

3. Experimental Results

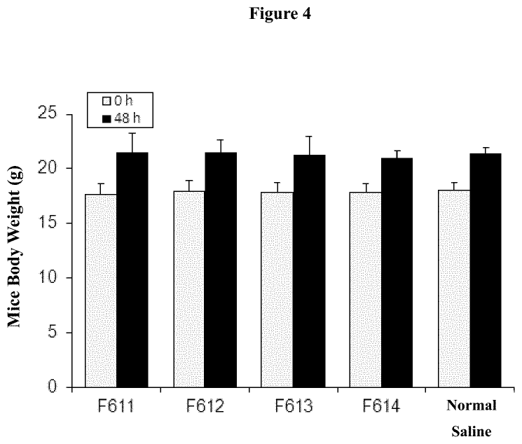

3.1 Variation of mice body weight. As shown in FIG. 4, no significant variation of mice body weight was shown in each group before and after administration, indicating that the liposome formulation as described herein had no significant toxicity.

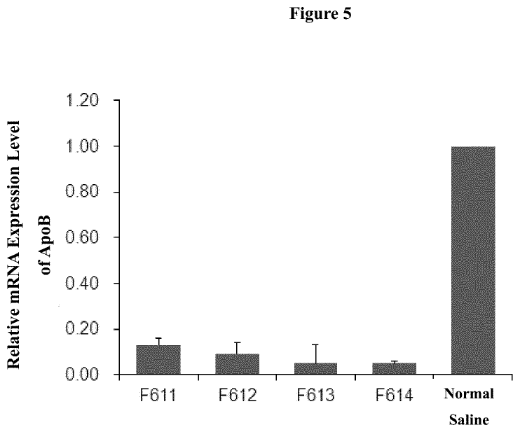

3.2 Variation of mRNA level of ApoB. As shown in FIG. 5, the liposome formation as described herein effectively inhibited expression of ApoB mRNA in liver.

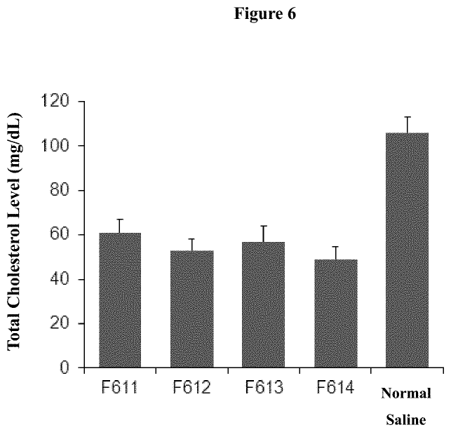

3.3 Variation of total cholesterol content. As shown in FIG. 6, in comparison with control group, the total cholesterol level in serum of mice was decreased by using the liposome formulation as described herein.

In sum, it can be seen that the liposome formulation of the present invention was able to deliver nucleic acid medicaments such as siRNA to target tissues or organs so as to facilitate the nucleic acid medicaments to play their role.

### EXAMPLE 5

1. Preparation of Liposome Formulation with siRNAs Encapsulated Therein

1.1 The cationic lipid compound consisted of lipid compound CL53 and cholesterol lipid compound CL68. Phospholipid was DSPC purchased from Shanghai Advanced Vehicle Technology L.T.D. Co. The PEGylated lipid was PEG-c-DMA.

1.2 The liposome formulation with siRNAs encapsulated therein was prepared according to the steps as described in Example 2. The lipid-type lipid compounds were listed in Table 5. Table 6 listed particle sizes and pKa values of the liposome formulations and the encapsulation efficiencies and loading efficiencies of the liposome formulations with siRNA encapsulated therein.

TABLE 5

| | Content | | | |
|---|---|---|---|---|
| | of cationic | Lipid-type lipd | | |
| | lipid | compound:Cholesterol | | Cholesterol |
| Formu- | compound | lipid | Lipid | lipid |
| lation | (%) | compound | compound | compound |
| F615 | 40 | 2:1 | CL53 | CL68 |
| F616 | 60 | 2:1 | CL53 | CL68 |
| F617 | 80 | 2:1 | CL53 | CL68 |
| F618 | 90 | 2:1 | CL53 | CL68 |

TABLE 6

| | | | siRNA | siRNA |
|---|---|---|---|---|
| | Particle Size | | Encapsulation | Loading |
| Formulation | (nm) | pKa value | Efficiencies | Efficiencies |
| F615 | 68.12 | 6.55 | 56.5 | 51.3 |
| F616 | 65.56 | 6.67 | 60.8 | 65.3 |
| F617 | 69.34 | 6.59 | 69.5 | 78.5 |
| F618 | 67.37 | 6.63 | 67.7 | 89.3 |

From the above table 6, it can be seen that siRNA encapsulation efficiency and loading efficiency were increased with increase of the content of cationic lipid compound in the liposome formulation. This indicated that the nucleic acid with negative charges loaded into the liposome was increased due to increase of the content of the cationic lipid compound. Therefore, the loading efficiency for nucleic acid medicaments was greatly increased by using the liposome formulation as described herein. As such, increased nucleic acid medicament was loaded into the unit lipid vesicle of the liposome formulation as described herein. At the same dosage, intake of cationic lipid compound was decreased due to increased loading amount, thereby reducing use of the lipid and toxicity.

2. Efficiency of the Liposome Formulation with siRNAs Encapsulated Therein

The efficiency of the liposome formulation with siRNAs encapsulated therein as prepared in this example was detected according to the method as described in Example 3.

3. Experimental Results

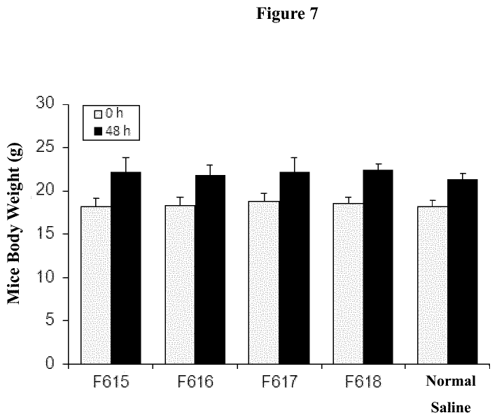

3.1 Variation of mice body weight. As shown in FIG. 7, no significant variation of mice body weight was shown in each group before and after administration, indicating that the liposome formulation as described herein had no significant toxicity.

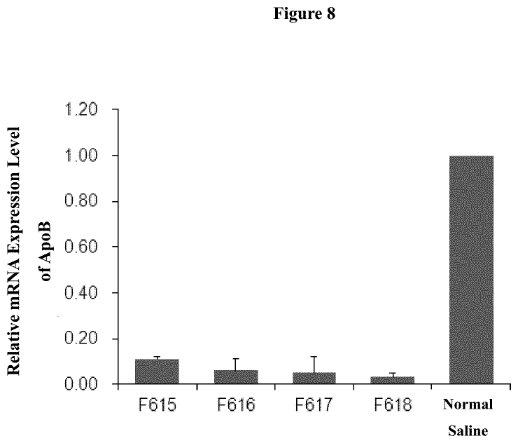

3.2 Variation of mRNA level of ApoB. As shown in FIG. 8, the liposome formation as described herein effectively inhibited expression of ApoB mRNA in liver.

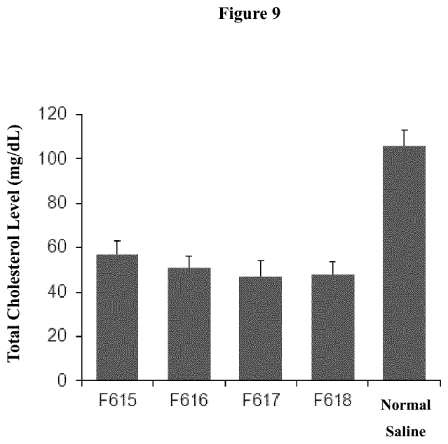

3.3 Variation of total cholesterol content. As shown in FIG. 9, in comparison with control group, the total cholesterol level in serum of mice was decreased by using the liposome formulation as described herein.

In sum, it can be seen that the liposome formulation of the present invention was able to deliver nucleic acid medicaments such as siRNA to target tissues or organs so as to facilitate the nucleic acid medicaments to play their role.

### EXAMPLE 6

1. Preparation of Liposome Formulation with siRNAs Encapsulated Therein

1.1 The cationic lipid compound consisted of lipid compound CL54 and cholesterol lipid compound CL68. Phospholipid was DSPC purchased from Shanghai Advanced Vehicle Technology L.T.D. Co. The PEGylated lipid was PEG-c-DMA.

1.2 The liposome formulation with siRNAs encapsulated therein was prepared according to the steps as described in Example 2. The ratios of the lipid-type lipid compound to the cholesterol lipid compound were listed in Table 7. Table 8 listed particle sizes and pKa values of the liposome formulations and the encapsulation efficiencies and loading efficiencies of the liposome formulations with siRNAs encapsulated therein.

TABLE 7

| | Content | | | |
|---|---|---|---|---|
| | of cationic | Lipid-type lipd | | |
| | lipid | compound:Cholesterol | | Cholesterol |
| Formu- | compound | lipid | Lipid | lipid |
| lation | (%) | compound | compound | compound |
| F619 | 40 | 2:1 | CL54 | CL68 |
| F620 | 60 | 2:1 | CL54 | CL68 |
| F621 | 80 | 2:1 | CL54 | CL68 |
| F622 | 90 | 2:1 | CL54 | CL68 |

TABLE 8

| | | | siRNA | siRNA |
|---|---|---|---|---|
| | | | Encapsulation | Loading |
| Formulation | Particle Size (nm) | pKa Value | Efficiency | Efficiency |
| F619 | 78.29 | 7.51 | 66.3 | 49.5 |
| F620 | 83.33 | 6.97 | 50.2 | 55.3 |
| F621 | 79.49 | 6.83 | 59.5 | 68.5 |
| F622 | 77.73 | 6.68 | 61.9 | 79.8 |

From the above table 8, it can be seen that siRNA encapsulation efficiency and loading efficiency were increased with increase of the content of cationic lipid compound in the liposome formulation. This indicated that the nucleic acid with negative charges loaded into the liposome was increased due to increase of the content of the cationic lipid compound. Therefore, the loading efficiency for nucleic acid medicaments was greatly increased by using the liposome formulation as described herein. As such, increased nucleic acid medicament was loaded into the unit lipid vesicles of the liposome formulation as described herein. At the same dosage, intake of cationic lipid compound was decreased due to increased loading amount, thereby reducing use of the lipid and toxicity.

2. Efficiency of the Liposome Formulation with siRNAs Encapsulated Therein

The efficiency of the liposome formulation with siRNAs encapsulated therein as prepared in this example was detected according to the method as described in Example 3.

3. Experimental Results

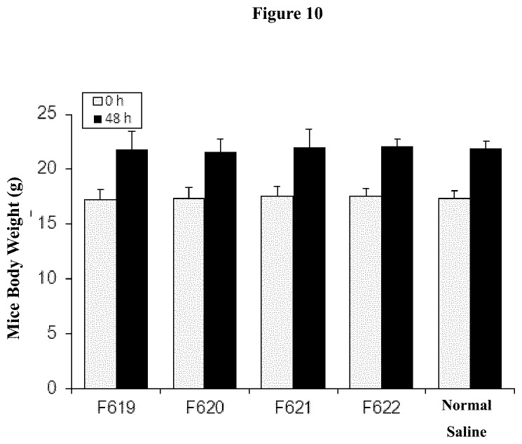

3.1 Variation of mice body weight. As shown in FIG. 10, no significant variation of mice body weight was shown in each group before and after administration, indicating that the liposome formulation as described herein had no significant toxicity.

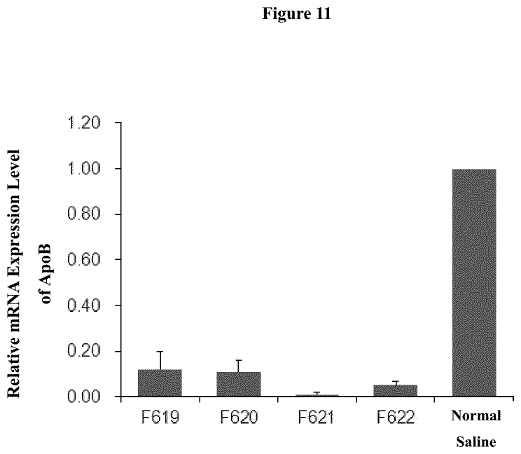

3.2 Variation of mRNA level of ApoB. As shown in FIG. 11, the liposome formation as described herein effectively inhibited expression of ApoB mRNA in liver.

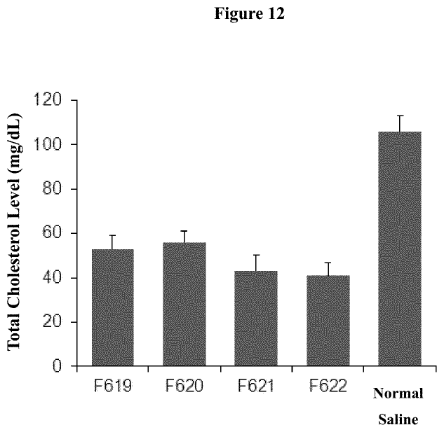

3.3 Variation of total cholesterol content. As shown in FIG. 12, in comparison with control group, the total cholesterol level in serum of mice was decreased by using the liposome formulation as described herein.

In sum, it can be seen that the liposome formulation of the present invention was able to deliver nucleic acid medicaments such as siRNA to target tissues or organs so as to facilitate the nucleic acid medicaments to play their role.

### EXAMPLE 7

1. Preparation of Liposome Formulation with siRNAs Encapsulated Therein

1.1 The cationic lipid compound consisted of lipid compound CL55 and cholesterol lipid compound CL68. Phospholipid was DSPC purchased from Shanghai Advanced Vehicle Technology L.T.D. Co. The PEGylated lipid was PEG-c-DMA.

1.2 The liposome formulation with siRNAs encapsulated therein was prepared according to the steps as described in Example 2. The ratios of the lipid-type lipid compound to the cholesterol lipid compound were listed in Table 9. Table 10 listed particle sizes and pKa values of the liposome formulations and the encapsulation efficiencies and loading efficiencies of the liposome formulations with siRNAs encapsulated therein.

TABLE 9

| | Content | | | |
|---|---|---|---|---|
| | of cationic | Lipid-type lipd | | |
| | lipid | compound:cholesterol | | cholesterol |
| | compound | lipid | Lipid | lipid |
| Formulation | (%) | compound | compound | compound |
| F623 | 40 | 2:1 | CL55 | CL68 |
| F624 | 60 | 2:1 | CL55 | CL68 |
| F625 | 80 | 2:1 | CL55 | CL68 |
| F626 | 90 | 2:1 | CL55 | CL68 |

TABLE 10

| | | | siRNA | siRNA |
|---|---|---|---|---|
| | | | Encapsulation | Loading |
| Formulation | Particle Size (nm) | pKa Value | Efficiency | Efficiency |
| F623 | 68.93 | 6.95 | 58.3 | 46.3 |
| F624 | 73.13 | 6.93 | 65.2 | 58.3 |
| F625 | 76.64 | 6.53 | 69.5 | 63.2 |
| F626 | 79.31 | 6.48 | 71.9 | 89.8 |

From the above table 10, it can be seen that siRNA encapsulation efficiency and loading efficiency were increased with increase of the content of cationic lipid compound in the liposome formulation. This indicated that the nucleic acid with negative charges loaded into the liposome was increased due to increase of the content of the cationic lipid compound. Therefore, the loading efficiency for nucleic acid medicaments was greatly increased by using the liposome formulation as described herein. As such, increased nucleic acid medicament was loaded into the unit lipid vesicle of the liposome formulation. At the same dosage, intake of cationic lipid compound was decreased due to increased loading amount, thereby reducing use of the lipid and toxicity.

2. Efficiency of the Liposome Formulation with siRNA Encapsulated Therein

The efficiency of the liposome formulation with siRNA encapsulated therein as prepared in this example was detected according to the method as described in Example 3.

3. Experimental Results

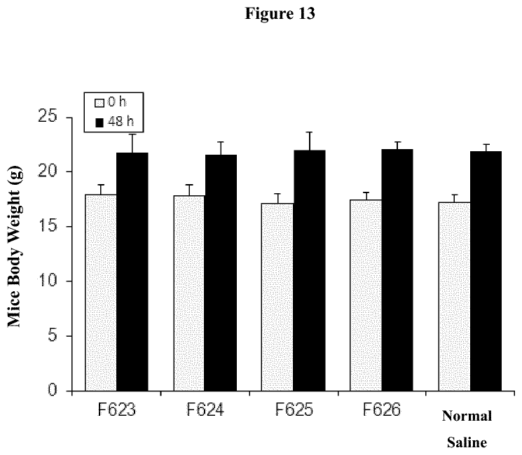

3.1 Variation of mice body weight. As shown in FIG. 13, no significant variation of mice body weight was shown in each group before and after administration, indicating that the liposome formulation as described herein had no significant toxicity.

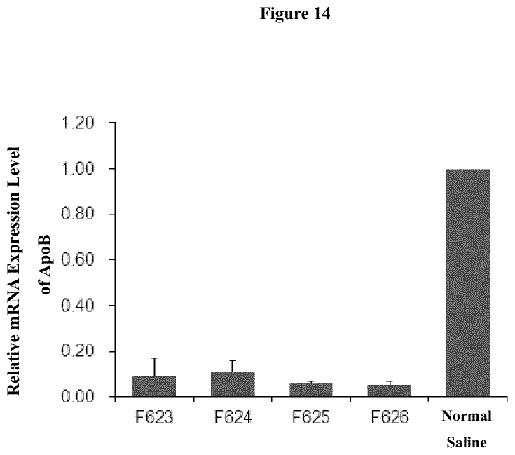

3.2 Variation of mRNA level of ApoB. As shown in FIG. 14, the liposome formation as described herein effectively inhibited expression of ApoB mRNA in liver.

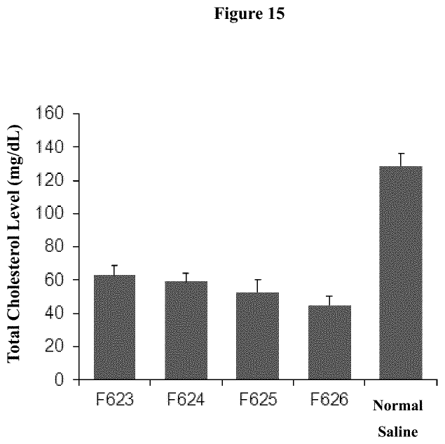

3.3 Variation of total cholesterol content. As shown in FIG. 15, in comparison with control group, the total cholesterol level in serum of mice was decreased by using the liposome formulation as described herein.

In sum, it can be seen that the liposome formulation of the present invention was able to deliver nucleic acid medicaments such as siRNA to target tissues or organs so as to facilitate the nucleic acid medicaments to play their role.

### EXAMPLE 8

1. Preparation of Liposome Formulation with siRNAs Encapsulated Therein

1.1 The cationic lipid compound consisted of lipid compound CL56 and cholesterol lipid compound CL68. Phospholipid was DSPC purchased from Shanghai Advanced Vehicle Technology L.T.D. Co. The PEGylated lipid was PEG-c-DMA.

1.2 The liposome formulation with siRNAs encapsulated therein was prepared according to the steps as described in Example 2. The ratios of the lipid-type lipid compound to the cholesterol lipid compound were listed in Table 11. Table 12 listed particle sizes and pKa values of the liposome formulations and the encapsulation efficiencies and loading efficiencies of the liposome formulations with siRNAs encapsulated therein.

TABLE 11

| | Content | | | |
|---|---|---|---|---|
| | of cationic | Lipid-type lipd | | |
| | lipid | compound:Cholesterol | | Cholesterol |
| Formu- | compound | lipid | Lipid | lipid |
| lation | (%) | compound | compound | compound |
| F627 | 40 | 2:1 | CL56 | CL68 |
| F628 | 60 | 2:1 | CL56 | CL68 |
| F629 | 80 | 2:1 | CL56 | CL68 |
| F630 | 90 | 2:1 | CL56 | CL68 |

TABLE 12

| | | | siRNA | siRNA |
|---|---|---|---|---|
| | | | Encapsulation | Loading |
| Formulation | Particle Size (nm) | pKa Value | Efficiency | Efficiency |
| F627 | 88.12 | 7.11 | 48.2 | 49.3 |
| F628 | 86.53 | 7.23 | 45.3 | 60.3 |
| F629 | 66.64 | 6.59 | 59.2 | 75.2 |
| F630 | 99.19 | 6.68 | 68.3 | 86.7 |

From the above table 12, it can be seen that siRNA encapsulation efficiency and loading efficiency were increased with increase of the content of cationic lipid compound in the liposome formulation. This indicated that the nucleic acid with negative charges loaded into the liposome was increased due to increase of the content of the cationic lipid compound. Therefore, the loading efficiency for nucleic acid medicaments was greatly increased by using the liposome formulation as described herein. As such, increased nucleic acid medicament was loaded into the unit lipid vesicle of the liposome formulation. At the same dosage, intake of cationic lipid compound was decreased due to increased loading amount, thereby reducing use of the lipid and toxicity.

2. Efficiency of the Liposome Formulation with siRNA Encapsulated Therein

The efficiency of the liposome formulation with siRNA encapsulated therein was detected according to the method as described in Example 3.

3. Experimental Results

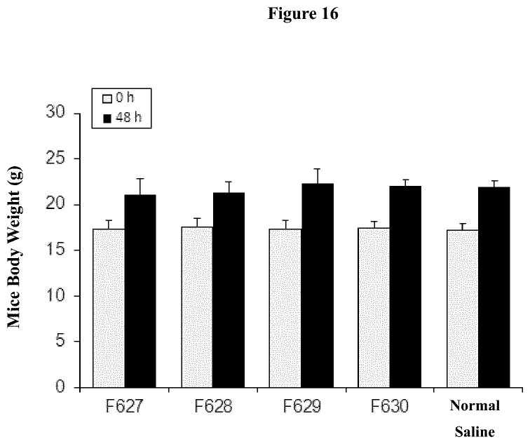

3.1 Variation of mice body weight. As shown in FIG. 16, no significant variation of mice body weight was shown in each group before and after administration, indicating that the liposome formulation as described herein had no significant toxicity.

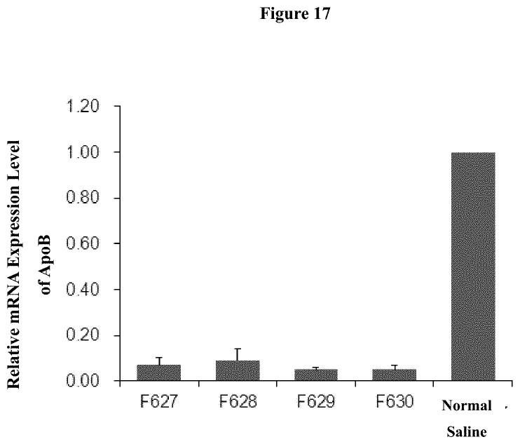

3.2 Variation of mRNA level of ApoB. As shown in FIG. 17, the liposome formation as described herein effectively inhibited expression of ApoB mRNA in liver.

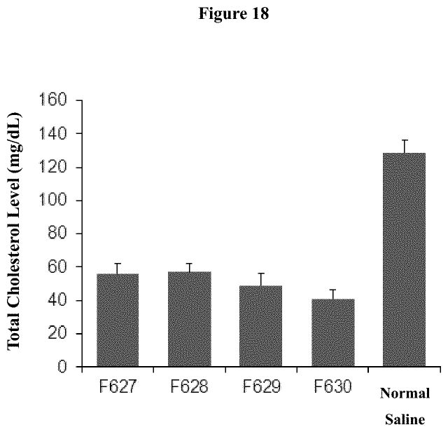

3.3 Variation of total cholesterol content. As shown in FIG. 18, in comparison with control group, the total cholesterol level in serum of mice was decreased by using the liposome formulation as described herein.

In sum, it can be seen that the liposome formulation of the present invention was able to deliver nucleic acid medicaments such as siRNA to target tissues or organs so as to facilitate the nucleic acid medicaments to play their role.

1. A liposome for delivery of nucleic acids, comprising a cationic lipid compound, a phospholipid and a PEGylated lipid, wherein the cationic lipid compound is in an amount from 50 wt % to 90 wt %.

2. The liposome of claim 1, wherein the cationic lipid compound is in an amount from 60 wt % to 90 wt %, or 70 wt % to 90 wt %, or 75 wt % to 90 wt %, or 80 wt % to 90 wt %, or 85 wt % to 90 wt %.

3. The liposome of claim 1 or 2, wherein the cationic lipid compound consists of a lipid-type lipid compound and a cholesterol lipid compound.

4. The liposome of claim 3, wherein the molar ratio of the lipid-type lipid compound to the cholesterol lipid compound ranges from 4:1 to 1:4.

5. The liposome of claim 1 or 2, wherein the phospholipid is in an amount from 5 wt % to 10 wt %.

6. The liposome of claim 1 or 2, wherein the PEGylated lipid is in an amount from 0 wt % to 10 wt %.

7. The liposome of claim 3, wherein the lipid-type lipid compound has a chemical structure selected from the group consisting of Formula I, Formula II, Formula III and Formula IV: 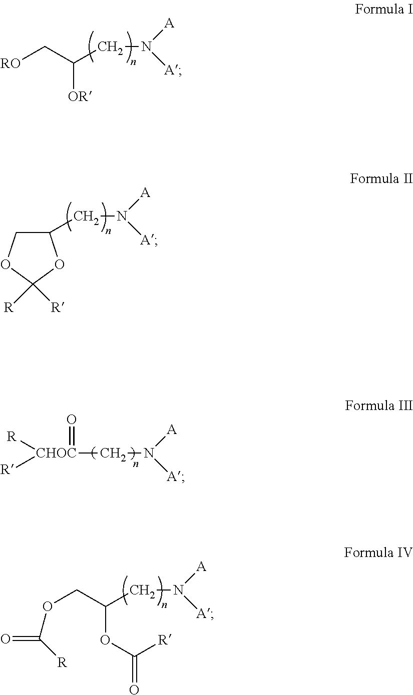
wherein, R and R′ are C₁₄-C₂₂ saturated or unsaturated fatty acids, A and A′ are C₁-C₄ alkyl, n=1-4.

8. The liposome of claim 3, wherein the cholesterol lipid compound has a chemical structure represented by Formula V: 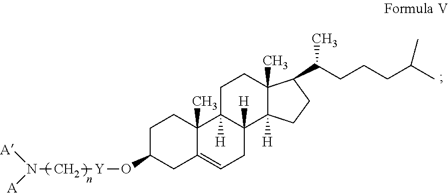
wherein, Y═(C═O), (C═S), (—HN(O)C—) or bond, A and A′ are C₁-C₄ alkyl, n=1-4.

9. A nucleic acid liposome formulation, comprising the liposome of any one of claims 1 to 8, and a nucleic acid encapsulated by the liposome.

10. The nucleic acid liposome formulation of claim 9, further comprising a pharmaceutically acceptable excipient.

11. The nucleic acid liposome formulation of claim 9 or 10, wherein, the formulation is in the form of tablet, capsule, lotion, drop, power, solution or aerosol.

12. The nucleic acid liposome formulation of claim 9 or 10, wherein the formulation is selected from the group consisting of an oral formulation, an intravascular injection formulation, an intramuscular injection formulation, a subcutaneous administration formulation, a parentenral administration formulation, and an intraperitoneal administration formulation.

13. The nucleic acid liposome formulation of claim 9, wherein the nucleic acid is selected from the group consisting of small interfering nucleic acid, micro nucleic acid, non-coding nucleic acid, antisense nucleic acid, small ligand nucleic acid and small active nucleic acid.

14. Use of the nucleic acid liposome formulation of any one of claims 9-13 in manufacturing a medicament for treating diseases associated with abnormal expression of genes.

15. A method for preparing the nucleic acid liposome formulation of any one of claims 9-13, comprising:
(1) providing a mixture solution of the cationic lipid compound, the phospholipid and the PEGylated lipid;
(2) mixing the said mixture solution and a nucleic acid solution to obtain the nucleic acid liposome formulation,
wherein the cationic lipid compound in the mixture solution are comprised from 50 wt % to 90 wt %.

16. The method of claim 15, wherein, after step (1), the said mixture solution is subject to extrusion process to obtain empty liposome vesicles.

17. The method of claim 16, wherein the nucleic acid solution is mixed with the empty liposome vesicles, such that the nucleic acid is loaded into the empty liposome vesicles to form the nucleic acid liposome formulation.
